(19) United States
(12) Patent Application Publication
(10) Pub. No.: US 2024/0383837 A1
(43) Pub. Date: Nov. 21, 2024

(54) METHOD FOR PRODUCING ACRYLIC ACID

(71) Applicant: LG CHEM, LTD., Seoul (KR)

(72) Inventors: Si Min KIM, Daejeon (KR); Donggyun KANG, Daejeon (KR); Woochul JUNG, Daejeon (KR); Yongbok JEONG, Daejeon (KR)

(73) Assignee: LG CHEM, LTD., Seoul (KR)

(21) Appl. No.: 18/689,591

(22) PCT Filed: May 31, 2023
(86) PCT No.: PCT/KR2023/007416

§ 371 Date: Mar. 6, 2024

(30) Foreign Application Priority Data
May 31, 2022 (KR) 10-2022-0066504
Oct. 7, 2022 (KR) 10-2022-0128680
May 30, 2023 (KR) 10-2023-0069400

(51) Int. Cl. C07C 51/44; C08J 11/16

(52) U.S. Cl. CPC C07C 51/44; C08J 11/16; C08J 2367/04

(57) ABSTRACT

The present invention provides a method for producing acrylic acid comprising thermally decomposing a poly(3-hydroxypropionate) in the presence of a predetermined transition metal oxide to produce acrylic acid.

### CROSS-REFERENCE TO RELATED APPLICATION(S)

This application a 35 U.S.C. § 371 National Stage of International Patent Application No. PCT/KR2023/007416, filed on May 31, 2023 which claims the benefit of Korean Patent Application No. 10-2022-0066504, filed on May 31, 2022, Korean Patent Application No. 10-2022-0128680, filed on Oct. 7, 2022, and Korean Patent Application No. 10-2023-0069400, filed on May 30, 2023, the disclosures of which are incorporated herein by reference in their entirety.

The present invention relates to a method for producing acrylic acid by thermally decomposing a poly (3-hydroxypropionate).

### BACKGROUND

Plastics are inexpensive and durable materials, which can be used to produce a variety of products that find use in a wide range of applications. As a consequence, the production of plastics has increased dramatically over the last decades. Moreover, 50% or more of such plastics are used for single-use disposable applications, such as packaging, agricultural films, disposable consumer articles, or for short-lived products that are discarded within a year of production. Because of the durability of the polymers, considerable quantities of plastics are piling up in landfill sites and in natural habitats worldwide, causing increasing environmental problems. Even degradable and biodegradable plastics may persist for decades depending on local environmental factors, such as levels of ultraviolet light exposure, temperature, presence of suitable microorganisms, etc.

Different solutions have been studied to reduce economic and environmental impacts correlated to the accumulation of plastic, from plastic degradation to plastic recycling.

As one example, polyethylene terephthalate (PET) is the most closed-loop recycled plastic (a system that processes and recycles waste from the production process). PET wastes (mainly bottles) are collected, sorted, pressed into bales, crushed, washed, chopped into flakes, melted and extruded in pellets and offered for sale. However, such plastic recycling methods are adapted to plastic articles containing only PET, and thus need a prior extensive sorting.

Another potential process for recycling plastic is chemical recycling allowing recovering the chemical constituents of the polymer. The resulting monomers may, after purification, be used to re-produce plastic articles.

On the other hand, 3-hydroxypropionic acid (3HP) is a platform compound that can be converted into various chemical substances such as acrylic acid, methyl acrylate, acrylamide, and the like. Since it was selected as one of the Top 12 value-added bio-chemicals by the US Department of Energy (DOE) in 2004, it has been actively studied in academia and industry. The production of such a 3-hydroxypropionic acid is largely made up of two methods of a chemical method and a biological method, but in the case of the chemical method, it is pointed out that it is not eco-friendly as the initial material is expensive and toxic substances are generated during the production process, so an eco-friendly bio-process is in the spotlight.

Further, 3-hydroxypropionic acid produced by eco-friendly bioprocesses is also polymerized or copolymerized, and used in disposable or short-term products, and there is a need for a chemical recycling method for recycling it.

### SUMMARY

### Technical Problem

It is an object of the present invention to provide a method for producing acrylic acid in high yield and high purity by thermally decomposing a poly(3-hydroxypropionate).

### Technical Solution

According to an embodiment of the present invention, there is provided a method for producing acrylic acid comprising thermally decomposing a poly(3-hydroxypropionate) in the presence of a transition metal oxide of one of Groups 5 to 12 of the periodic table of elements to produce acrylic acid.

Now, a method for producing acrylic acid according to specific embodiments of the invention will be described in more detail.

Unless particularly mentioned herein, the term “including” or “comprising” refers to including some element (or component) without any limitation, and should not be construed as excluding addition of other elements (or components).

In addition, it should be understood that, unless steps included in a production method described herein are specified as being sequential or consecutive or otherwise stated, one step and another step included in the production method should not be construed as being limited to an order described herein. Therefore, it should be understood that an order of steps included in a production method can be changed within the range of understanding of those skilled in the art, and that, in this case, the incidental changes obvious to those skilled in the art are within the scope of the present invention.

Further, unless otherwise stated herein, the weight average molecular weight of poly(3-hydroxypropionate) can be measured using a gel permeation chromatography(GPC). Specifically, the polymer is dissolved in chloroform to a concentration of 2 mg/ml, then 20 μl is injected into GPC, and GPC analysis is performed at 40° C. At this time, chloroform is used as the mobile phase for GPC, the flow rate is 1.0 mL/min, and the column used is two Agilent Mixed-B units connected in series. RI Detector is used as a detector. The values of Mw can be obtained using a calibration curve formed using a polystyrene standard sample. Nine kinds of the polystyrene standard samples are used with the molecular weight of 2,000 g/mol, 10,000 g/mol, 30,000 g/mol, 70,000 g/mol, 200,000 g/mol, 700,000 g/mol, 2,000,000 g/mol, 4,000,000 g/mol, and 10,000,000 g/mol.

According to one embodiment of the invention, there is provided a method for producing acrylic acid comprising thermally decomposing a poly(3-hydroxypropionate) in the presence of a transition metal oxide of one of Groups 5 to 12 of the periodic table of elements to produce acrylic acid.

The present inventors have found that when poly(3-hydroxypropionate), for example, poly(3-hydroxypropionate) formed by polymerizing 3-hydroxypropionic acid produced by fermenting a strain having 3-hydroxypropionic acid production ability, is thermally decomposed in the presence of a transition metal oxide of one of Groups 5 to 12 of the periodic table of elements, acrylic acid can be recovered in high purity and yield even at low thermal decomposition temperatures, thereby reducing energy costs, and completed the present invention.

Also, the poly(3-hydroxypropionate) is thermally decomposed and recycled as a recyclable monomer, thereby being eco-friendly and economical, and the acrylic acid can be recycled into bio-superabsorbent polymer (SAP) or bioacrylate.

The method for producing acrylic acid according to one embodiment may, before the thermally decomposing a poly(3-hydroxypropionate) in the presence of a transition metal oxide of one of Groups 5 to 12 of the periodic table of elements to produce acrylic acid, further comprise melting the poly(3-hydroxypropionate).

That is, the poly(3-hydroxypropionate) can be melted before the thermal decomposition, and this melting process can be performed in the presence of the transition metal oxide of one of Groups 5 to 12 of the periodic table of elements. When the melting process is performed under transition metal oxide, the method may, before the melting step, further comprise adding poly(3-hydroxypropionate) with a transition metal oxide of one of Groups 5 to 12 of the periodic table of elements to a reactor and mixing them.

Before thermal decomposition of the poly(3-hydroxypropionate), volatile substances remaining due to the melting and/or impurities flowing in during recycling can be removed. Further, the mobility of the copolymer can be increased due to the melting, so that it can be easily added to a thermal decomposition reactor, and by utilizing this point, thermal decomposition can be continuously proceeded in the future.

The melting may be performed at a temperature of 150° C. or more and 200° C. or less. For example, after mixing poly(3-hydroxypropionate) with a transition metal oxide of one of Groups 5 to 12 of the periodic table of elements in the reactor, the poly(3-hydroxypropionate) can be melted at a temperature of 150° C. or more and 200° C. or less.

Further, the temperature during melting may be 150° C. or more, 160° C. or more, or 170° C. or more, and 200° C. or less, 190° C. or less, or 180° C. or less. If the melting temperature is too low, the poly(3-hydroxypropionate) is not melted, residual volatile substances and/or impurities flowing in during recycling cannot be removed, and it may be difficult to proceed continuous thermal decomposition. If the melting temperature is too high, the poly(3-hydroxypropionate) is thermally decomposed without melting, which reduces the recovery rate of bio-acrylic acid, or large amounts of impurities may flow in because there is no process of removing impurities before thermal decomposition, and bumping or the like due to a sudden temperature rise may be generated.

On the other hand, the melting may be performed under a solvent-free condition. For example, when the melting is performed under a solvent-free condition, the poly(3-hydroxypropionate) may be melted in a state that is not dissolved in a solvent. When a solvent is added to the reactor, impurities may be formed in the process of thermally decomposing the poly(3-hydroxypropionate), and a solvent removal process and an additional impurity removal process are required, which may complicate the process or require additional equipment. Further, side reactions may occur during the process of removing the solvent, which may result in a decrease in the recovery rate and purity of monomers. In addition, there is a disadvantage in that economic efficiency is lowered due to the use of additional solvents.

Further, in the method for producing acrylic acid according to one embodiment, the poly(3-hydroxypropionate) melted by the melting may have a complex viscosity of 5.0 Pa·s or more and 30.0 Pa·s or less at an angular frequency of 0.1 to 500.0 rad/s. For example, the melted poly(3-hydroxypropionate) may have a complex viscosity of 5.0 Pa·s or more and 30.0 Pa·s or less, 7.0 Pa·s or more and 28.0 Pa·s or less, 9.0 Pa·s or more and 26.0 Pa·s or less, 11.0 Pa·s or more and 24.0 Pa·s or less at an angular frequency of 0.1 to 500.0 rad/s.

In the method for producing acrylic acid according to one embodiment, acrylic acid can be produced by thermally decomposing the melted poly(3-hydroxypropionate).

Because the thermal decomposition of the melted poly(3-hydroxypropionate) is performed in the presence of a transition metal oxide of one of Groups 5 to 12 of the periodic table of elements, the thermal decomposition occurs at a low temperature of 250° C. or less, which reduces the thermal decomposition energy, reduces the possibility of forming impurities due to high heat, and allows the thermal decomposition to occur faster, thereby improving production quantity per reaction time.

On the other hand, the thermal decomposition may be performed at a temperature of 200° C. or more and 250° C. or less, and for example, the temperature may be 200° C. or more, 205° C. or more, 210° C. or more, or 215° C. or more, and 250° C. or less, or 240° C. or less. If the thermal decomposition temperature is too low, the thermal decomposition of the poly(3-hydroxypropionate) may not occur, and if the thermal decomposition temperature is too high, the operating cost of the thermal decomposition process may increase, or large amounts of unexpected impurities may be generated.

Further, the thermal decomposition may be performed at a pressure of more than 0.01 torr and 50 torr or less, and for example, the pressure may be more than 0.01 torr, 0.05 torr or more, 0.1 torr or more, 0.5 torr or more, 1 torr or more, 2 torr or more, 4 torr or more, or 5 torr or more, and, 50 torr or less, 40 torr or less, 30 torr or less, or 20 torr or less, but is not limited thereto. As the thermal decomposition pressure is lower, the separation and recovery of the final produced bio-acrylic acid can be facilitated.

In the method for producing acrylic acid according to one embodiment, the difference between the melting temperature and the thermal decomposition temperature may be 20° C. or more and 130° C. or less, 30° C. or more and 120° C. or less, 40° C. or more and 110° C. or less, 50° C. or more and 100° C. or less, or 60° C. or more and 90° C. or less. Because the difference between the melting temperature and the thermal decomposition temperature is small, that is, because the melting temperature is high and the thermal decomposition temperature is low, a continuous thermal decomposition process is facilitated, energy saving effects are achieved, and problems such as bumping due to a sudden temperature rise between melting and thermal decomposition processes can be prevented.

Moreover, the thermal decomposition can be performed under a solvent-free condition. For example, when the thermal decomposition is performed under a solvent-free condition, the poly(3-hydroxypropionate) may be thermally decomposed in a state that is not dissolved into a solvent. When a solvent is added to the reactor, impurities may be formed in the process of thermally decomposing the poly(3-hydroxypropionate), and a solvent removal process and an additional impurity removal process are required, which may complicate the process or require additional equipment. Further, side reactions may occur during the process of removing the solvent, which may result in a decrease in the recovery rate and purity of monomers. Further, there is a disadvantage in that economic efficiency is lowered due to the use of additional solvents.

Further, in the method for producing acrylic acid according to one embodiment, by using a transition metal oxide of one of Groups 5 to 12 of the periodic table of elements, the thermal decomposition temperature of the poly(3-hydroxypropionate) can be lowered to reduce energy costs. For example, as the transition metal oxides of one of Groups 5 to 12 of the periodic table of elements is used, the thermal decomposition temperature of poly(3-hydroxypropionate) may be 250° C. or less, 245° C. or less, or 240° C. or less.

In addition, the transition metal oxide remains in a solid phase even after the thermal decomposition process, which makes it easy to separate from the acrylic acid that is finally produced, and thus, the acrylic acid can be recovered with high recovery rate and high purity.

The transition metal oxide of one of Groups 5 to 12 of the periodic table of elements may be, for example, one or more selected from the group consisting of zinc oxide, iron oxide, copper oxide, nickel oxide, cobalt oxide, manganese oxide, chromium oxide, and molybdenum oxide. Further, the temperature at which the weight of poly(3-hydroxypropionate) begins to decrease (weight loss temperature) is about 295° C. In order to lower the thermal decomposition temperature of poly(3-hydroxypropionate) to 250° C. or less, it is preferable to use zinc oxide, which can dramatically reduce the thermal decomposition energy.

Further, the transition metal oxide of one of Groups 5 to 12 of the periodic table of elements may be used in an amount of 0.01 parts by weight or more, 0.10 parts by weight or more, 0.50 parts by weight or more, or 1.00 parts by weight or more, and 30 parts by weight or less, 25 parts by weight or less, 20 parts by weight or less, 15 parts by weight or less, and 10 parts by weight or less, based on 100 parts by weight of the poly(3-hydroxypropionate). If the amount of the transition metal oxide added is too small, thermal decomposition of poly(3-hydroxypropionate) may not occur, and if the amount of the transition metal oxide added is too large, the economic efficiency may deteriorate due to the excessive addition.

The method for producing acrylic acid may recycle biodegradable articles comprising the poly(3-hydroxypropionate).

The poly(3-hydroxypropionate) is a biodegradable compound, and a biodegradable article containing the same is not particularly limited as long as it is a general article containing biodegradable plastic, but an example thereof may be a plastic bag, fiber, fabric, food container, toothbrush, film, fishing net, or packaging material. Further, the method for producing acrylic acid according to one embodiment can thermally decompose a biodegradable article containing such a poly(3-hydroxypropionate) to recover an acrylic acid monomer, thereby recycling the biodegradable article. In addition, monomers such as acrylic acid recovered through thermal decomposition can be polymerized to produce a biodegradable plastic, thereby recycling as biodegradable articles or the like.

Acrylic acid produced in the method for producing acrylic acid according to one embodiment may include a radioactive carbon isotope (¹⁴C).

The radioactive carbon isotope (¹⁴C) contains about 1 atom per 10¹² carbon atoms in the earth's atmosphere, and has a half-life of about 5700 years. The stock of carbon can become abundant in the upper atmosphere by a nuclear reaction involving cosmic rays and ordinary nitrogen (¹⁴N). Meanwhile, in fossil fuels, isotopes have decayed over a long time, so that the ¹⁴C ratio may be substantially zero. When bio-derived 3-hydroxypropionic acid is used as a raw material for poly(3-hydroxypropionate) or fossil fuel is used together with it, the content of radioactive carbon isotope (pMC; percent modern carbon) and the content of biocarbon contained in the bio acrylic acid can be measured according to the standard of ASTM D6866-21.

As an example of the measurement method, the carbon atoms contained in the compound to be measured can be made into graphite or carbon dioxide gas, and the content can be measured using a mass spectrometer, or can be measured according to a liquid scintillation analysis method. At this time, the two radioactive isotopes can be separated using an accelerator for separating ¹⁴C ions from ¹²C ions together with the mass spectrometer, and the content and the content ratio can be measured by a mass spectrometer.

The acrylic acid may have a radioactive carbon isotope content of 20 pMC (percent modern carbon) or more, 50 pMC or more, 90 pMC or more, or 100 pMC or more as measured according to the standard of ASTM D6866-21. The acrylic acid may also have a biocarbon content of 80 wt. % or more, 85 wt. % or more, 90 wt. % or more, 95 wt. % or more, or 100 wt. % as measured according to the standard of ASTM D6866-21.

The radioactive carbon isotope ratio (pMC) means the ratio between the radioactive carbon isotope (¹⁴C) contained in the acrylic acid and the radioactive carbon isotope (¹⁴C) of a modern reference standard material, and it can be greater than 100% because the nuclear testing program in the 1950s is still in effect and is not extinguished.

Further, the content of biocarbon means the content of biocarbon relative to the total carbon content contained in the acrylic acid. As this value is larger, it may correspond to an eco-friendly compound.

On the other hand, if the radioactive carbon isotope content (pMC) and biocarbon content of the acrylic acid are too low, eco-friendly property is reduced, which may not be seen as a bio-derived material.

In the method for producing acrylic acid according to one embodiment, after the step of thermally decomposing poly(3-hydroxypropionate) to produce acrylic acid, the acrylic acid may be recovered by distillation under reduced pressure.

For example, after the thermal decomposition process, acrylic acid produced by reducing the pressure to more than 1 torr can be recovered by distillation under reduced pressure. Further, acrylic acid can be recovered by distillation even under normal pressure conditions.

At this time, the recovery rate of acrylic acid may be 30% or more, 50% or more, 60% or more, 70% or more, 80% or more, or 90% or more, for example, 40 to 99.9%, 50 to 99.9%, 60 to 99.9%, 70 to 99.9%, 80 to 99.9%, 90 to 99.9%, 40 to 99%, 50 to 99%, 60 to 99%, 70 to 99%, 80 to 99%, 90 to 99%, 40 to 97%, 50 to 97%, 60 to 97%, 70 to 97%, 80 to 97%, 90 to 97%, 40 to 95%, 50 to 95%, 60 to 95%, 70 to 95%, 80 to 95%, or 90 to 95%. The recovery rate may be calculated on a weight or mole basis.

The purity of acrylic acid may be 50% or more, 60% or more, 70% or more, 80% or more, or 90% or more, for example, 40 to 99.9%, 50 to 99.9%, 60 to 99.9%, 70 to 99.9%, 80 to 99.9%, 90 to 99.9%, 40 to 99%, 50 to 99%, 60 to 99%, 70 to 99%, 80 to 99%, 90 to 99%, 40 to 97%, 50 to 97%, 60 to 97%, 70 to 97%, 80 to 97%, 90 to 97%, 40 to 95%, 50 to 95%, 60 to 95%, 70 to 95%, 80 to 95%, or 90 to 95%.

The method for producing acrylic acid according to one embodiment may further comprise a step of polymerizing 3-hydroxypropionic acid to produce the poly(3-hydroxypropionate).

The step of polymerizing 3-hydroxypropionic acid to produce the poly(3-hydroxypropionate) may be performed before the step of melting the poly(3-hydroxypropionate).

The 3-hydroxypropionic acid may be produced by fermenting a strain having 3-hydroxypropionic acid production ability, and when the poly(3-hydroxypropionate) polymerized with 3-hydroxypropionic acid is thermally decomposed as described above, acrylic acid, which is a recyclable monomer, can be recovered in high purity and high yield.

Further, the 3-hydroxypropionic acid can be produced through the following steps: (step 1) fermenting a strain having 3-hydroxypropionic acid production ability to produce a 3-hydroxypropionic acid fermented liquid; (step 2) concentrating the fermented liquid in the presence of an alkali metal salt to form a 3-hydroxypropionate crystal; and (step 3) separating the 3-hydroxypropionate crystal and converting it into 3-hydroxypropionic acid.

The strain having 3-hydroxypropionic acid production ability may include a gene encoding at least one or two proteins selected from the group consisting of glycerol dehydratase and aldehyde dehydrogenase.

In one example, the 3-hydroxypropionic acid producing strain may further include a gene (gdrAB) encoding glycerol dehydratase reactivase (GdrAB). In one example, the 3-hydroxypropionic acid producing strain may be a strain that is capable of further biosynthesizing vitamin B₁₂.

The glycerol dehydratase may be encoded by the dhaB (GenBank accession no. U30903.1) gene, but is not limited thereto. The dhaB gene may be an enzyme derived from Klebsiella pneumoniae, but is not limited thereto. The gene encoding glycerol dehydratase may include genes encoding dhaB1, dhaB2, and/or dhaB3. The glycerol dehydratase protein and the gene encoding it may include mutations in the gene and/or amino acid sequence within the range that maintains enzymatic activity that degrades glycerol to 3-hydroxypropanal (3-HPA) and water (H₂O).

The gene (aldH) encoding the aldehyde dehydrogenase (ALDH) may be, for example, aldH (GenBank Accession no. U00096.3; EaldH) gene from Escherichia coli or E. coli K12 MG1655 cell line; puuC gene from Klebsiella pneumoniae (K. pneumonia), and/or KGSADH gene from Azospirillum brasilense, but is not limited thereto. The aldehyde dehydrogenase protein and the gene encoding it may include mutations in the gene and/or amino acid sequence within a range that maintains the activity for producing 3-hydroxypropionic acid from 3-hydroxypropanal.

The medium for producing the fermentation liquid can be selected without limitation within the scope of the purpose for producing 3-hydroxypropionic acid. In one example, the medium may include glycerol as a carbon source. In another example, the medium may be crude glycerol and/or pretreated waste glycerol, but is not limited thereto. In one example, the production medium may further include vitamin B₁₂.

In the step of fermenting the strain having 3-hydroxypropionic acid production ability to produce a 3-hydroxypropionic acid fermentation liquid, the concentration of 3-hydroxypropionic acid contained in the 3-hydroxypropionic acid fermentation liquid may be 1 to 200 g/L, 10 to 150 g/L, 30 to 130 g/L, or 40 to 100 g/L.

Further, the fermentation may be a neutral fermentation, and for example, the pH during fermentation may be maintained within the range of 6 to 8, 6.5 to 8, 6 to 7.5, or 6.5 to 7.5, but is not limited thereto. The pH range can be appropriately adjusted as necessary. An alkali metal salt may be added for the neutral fermentation. The alkali metal salt may include Mg²⁺, Ca²⁺, or a mixture thereof. Further, the alkali metal salt may be Ca(OH)₂ or Mg(OH)₂, but is not limited thereto.

After producing the 3-hydroxypropionic acid fermentation liquid, the method may further comprise removing (separating) cells from the fermentation liquid; purifying and/or decolorizing the fermentation liquid and/or the fermentation liquid from which cells have been removed; and/or filtering the fermentation liquid and/or the fermentation liquid from which cells have been removed.

Removal (separation) of the cells can be performed by selecting a method known in the art, without limitation, within the scope of the purpose of cell (strain) removal. In one example, removal of the cells may be performed by centrifugal separation.

The step of purifying and/or decolorizing the fermentation liquid and/or the fermentation liquid from which cells have been removed may be performed by selecting a method known in the art without limitation within the scope of the purpose for purifying the fermentation liquid, and it may be performed, for example, by mixing activated carbon with the fermentation liquid and then removing the activated carbon, but is not limited thereto.

The step of filtering the fermentation liquid and/or the fermentation liquid from which cells have been removed may be performed by selecting a method known in the art without limitation within the scope of the purpose for removing solid impurities, removing proteins and/or substances with hydrophobic functional groups, and/or decolorizing the liquid. For example, it may be performed by filter filtration and/or activated carbon filtration methods, but is not limited thereto.

Further, after the step of fermenting the strain having 3-hydroxypropionic acid production ability to produce a 3-hydroxypropionic acid fermentation liquid, the fermentation liquid can be concentrated in the presence of an alkali metal salt to form crystals of 3-hydroxypropionate.

Concentration of the fermentation liquid may be performed by evaporating the fermentation liquid (e.g., liquid component of the fermentation liquid).

The concentration can be performed by any means commonly available for evaporating the liquid components of the fermentation liquid, and for example, it can be performed by rotary evaporation concentration, evaporation concentration, vacuum concentration, reduced pressure concentration, and the like, but is not limited thereto.

In one example, the concentration of 3-hydroxypropionic acid in the concentrated fermentation liquid may be increased by 2 to 50 times, 2 to 40 times, 2 to 30 times, 2 to 20 times, 2 to 10 times, 5 to 50 times, 5 to 40 times, 5 to 30 times, 5 to 20 times, or 5 to 10 times compared to before concentration.

The fermentation liquid may be concentrated so that the fermentation liquid contains 300 g/L or more of 3-hydroxypropionic acid. For example, the concentrate containing 3-hydroxypropionic acid may contain 3-hydroxypropionic acid at a concentration of 300 g/L or more, 350 g/L or more, 400 g/L or more, 450 g/L or more, and 500 g/L or more, and may contain 3-hydroxypropionic acid at a concentration of 900 g/L or less, 850 g/L or less, or 800 g/L or less.

Whether to form the 3-hydroxypropionic acid crystal appears to be affected by the presence of an alkali metal salt, the concentration of 3-hydroxypropionic acid in the concentrate, and the like.

Further, when the concentration of 3-hydroxypropionic acid crystals in the concentrate is higher than the water solubility of the 3-hydroxypropionic acid crystals, the 3-hydroxypropionic acid crystals can be produced more easily.

For example, the water solubility of Ca(3HP)₂, which is the 3-hydroxypropionic acid crystal, is 450 g/L at room temperature and thus, when the concentration of 3-hydroxypropionic acid in the concentrate exceeds 450 g/L, the formation of Ca(3HP)₂ crystal may be promoted. Further, the water solubility of Mg(3HP)₂, which is the crystal of 3-hydroxypropionic acid, is 250 g/L at room temperature, and thus, when the concentration of 3-hydroxypropionic acid in the concentrate exceeds 250 g/L, the formation of Mg(3HP)₂ crystals may be promoted.

The 3-hydroxypropionate crystal can be formed from a concentrate that contains the alkali metal salt and satisfies the above-mentioned concentration, and the alkali metal salt may be selected without limitation within the scope of the purpose for forming crystals of 3-hydroxypropionate. For example, the alkali metal salt may include one or more cations selected from the group consisting of Na⁺, Mg²⁺, and Ca²⁺. Further, the alkali metal salt may be Ca(OH)₂, Mg(OH)₂, or a mixture thereof.

The alkali metal salt is added and remains in the process of producing the 3-hydroxypropionic acid fermentation liquid, or may be added during the process of forming 3-hydroxypropionate crystal in a concentrate containing 300 g/L or more of 3-hydroxypropionic acid. Further, the concentration of the alkali metal salt may be 10% to 100%, or 30% to 90% of the concentration of 3-hydroxypropionic acid, and for example, it may be present in the concentrate at a concentration of 10 to 900 g/L, 50 to 800 g/L, 100 to 700 g/L, or 200 to 600 g/L.

The 3-hydroxypropionate crystal may be in the form of Structural Formula 1 or Structural Formula 2 below. That is, the 3-hydroxypropionate crystal may include 3-hydroxypropionate in the form of Structural Formula 1 or Structural Formula 2 below.

In Structural Formula 1 and Structural Formula 2 below, Cation means a cation, 3HP means 3-hydroxypropionic acid that binds to the cation, n is the number of 3HP that binds to the cation, and means an integer of 1 or more, and in Structural Formula 2 below, m is the number of water molecules that binds to Cation(3HP)n in the hydrate, which is an integer of 1 or more. The cation may be, for example, Na⁺, Mg²⁺, or Ca²⁺, but is not limited thereto.

Cation(3HP)ₙ [Structural Formula 1]

Cation(3HP)ₙ·mH₂O [Structural Formula 2]

Further, the step of forming 3-hydroxypropionate crystal may further include stirring the concentrate.

The stirring step may be performed at a temperature of 0 to 70 degrees Celsius, 0 to 60 degrees Celsius, 0 to 50 degrees Celsius, 0 to 40 degrees Celsius, 0 to 35 degrees Celsius, 0 to 30 degrees Celsius, 10 to 70 degrees Celsius, 10 to 60 degrees Celsius, 10 to 50 degrees Celsius, 10 to 40 degrees Celsius, 10 to 35 degrees Celsius, 10 to 30 degrees Celsius, 15 to 70 degrees Celsius, 15 to 60 degrees Celsius, 15 to 50 degrees Celsius, 15 to 40 degrees Celsius, 15 to 35 degrees Celsius, 15 to 30 degrees Celsius, 20 to 70 degrees Celsius, 20 to 60 degrees Celsius, 20 to 50 degrees Celsius, 20 to 40 degrees Celsius, 20 to 35 degrees Celsius, or 20 to 30 degrees Celsius (i.e., room temperature), and/or under conditions of 100 to 2000 rpm, 100 to 1500 rpm, 100 to 1000 rpm, 100 to 500 rpm, 100 to 400 rpm, or 200 to 400 rpm (e.g., about 300 rpm).

The particle size distribution D₅₀ of the 3-hydroxypropionate crystal may be 20 μm or more and 90 μm or less, 25 μm or more and 85 μm or less, 30 μm or more and 80 μm or less, or 35 μm or more and 75 μm or less.

Further, the particle size distribution D₁₀ of the 3-hydroxypropionate crystal may be 5 ηm or more and 40 μm or less, 8 μm or more and 35 μm or less, and 10 μm or more and 30 μm or less, and the particle size distribution D₉₀ of the 3-hydroxypropionate crystal may be 50 μm or more and 200 μm or less, 60 μm or more and 190 μm or less, 65 μm or more and 180 μm or less, and 70 μm or more and 175 μm or less.

The particle size distributions D₅₀, D₁₀ and D₉₀ mean particle sizes at which cumulative volumes of particles reach 50%, 10% and 90%, respectively, in the particle size distribution curve of the particles, wherein the D₅₀, D₁₀ and D₉₀ can be measured using, for example, a laser diffraction method. The laser diffraction method can generally measure particle sizes ranging from a submicron range to several millimeters, and can obtain results with high reproducibility and high resolvability.

If the particle size distributions D₅₀, D₁₀ and D₉₀ of the 3-hydroxypropionate crystal is too large, impurities that must be removed during crystallization may be contained in the crystals, which results in a reduction in purification efficiency. If the particle size distributions are too small, the liquid permeability during filtration of the crystals may be lowered.

Meanwhile, the (D₉₀−D₁₀)/D₅₀ of the 3-hydroxypropionate crystal may be 1.00 or more and 3.00 or less, 1.20 or more and 2.80 or less, 1.40 or more and 2.60 or less, or 1.60 or more and 2.40 or less.

Further, the 3-hydroxypropionate crystal may have a volume average particle size of 30 μm or more and 100 μm or less, 35 μm or more and 95 μm or less, or 40 μm or more and 90 μm or less, a number average particle size of 1 μm or more and 30 μm or less, 3 μm or more and 25 μm or less, or 5 μm or more and 20 μm or less, and a volume average particle size of 10 μm or more and 70 μm or less, 15 μm or more and 60 μm or less, or 20 μm or more and 55 μm or less.

If the volume average particle size, number average particle size, and volume average particle size of the 3-hydroxypropionate crystal are too large, impurities that must be removed during crystallization may be contained in the crystals, which results in a reduction in purification efficiency. If they are too small, the liquid permeability during filtration of the crystals may be lowered.

Further, the LW ratio (length to width ratio) and average LW ratio in the particle size distribution (D₁₀, D₅₀, D₉₀) of the 3-hydroxypropionate crystal are 0.50 or more and 3.00 or less, 0.70 or more, 2.80 or less, and 1.00 or more and 2.50 or less. If the LW ratio of the 3-hydroxypropionate crystal is too large, problems of fluidity and clogging may occur during transfer of the crystals, and if the LW ratio is too small, the liquid permeability during filtration of the crystals may be lowered.

The 3-hydroxypropionate crystal can measure the moisture content contained in the crystals by the Karl Fischer method, and the moisture content contained in the 3-hydroxypropionate crystal may be 200 ppm or more and 5000 ppm or less, 250 ppm or more and 4800 ppm or less, 300 ppm or more and 4600 ppm or less, or 350 ppm or more and 4400 ppm or less.

At this time, the moisture contained in the 3-hydroxypropionate crystal means the attached moisture contained between the crystals, rather than the crystal moisture (for example, Ca(3HP)₂·2H₂O). Further, if the moisture content contained in the 3-hydroxypropionate crystal is too high, it may be recovered in the form of a slurry rather than a crystalline solid, or impurities may be contained in the water, which may cause a problem of a decrease in purity improvement.

The 3-hydroxypropionate crystal may contain a radioactive carbon isotope (¹⁴C).

The radioactive carbon isotope (¹⁴C) contains about 1 atom per 10¹² carbon atoms in earth's atmosphere and has a half-life of about 5700 years, and the stock of carbon can become abundant in the upper atmosphere due to nuclear reactions in which cosmic rays and normal nitrogen (¹⁴N) participate. Meanwhile, in fossil fuels, isotopes have decayed over a long time, so that the ¹⁴C ratio may be substantially zero. When bio-derived raw materials are used as the 3-hydroxypropionic acid raw material, or fossil fuels are used together with it, the content of radioactive carbon isotopes (pMC; percent modern carbon) and the content of bio-carbon contained in 3-hydroxypropionic acid can be measured according to the standard of ASTM D6866-21.

As an example of the measurement method, the carbon atoms contained in the compound to be measured can be made into graphite or carbon dioxide gas, and the content can be measured using a mass spectrometer, or can be measured according to a liquid scintillation analysis method. At this time, the two radioactive isotopes can be separated using an accelerator for separating ¹⁴C ions from ¹²C ions together with the mass spectrometer, and the content and the content ratio can be measured by a mass spectrometer.

The 3-hydroxypropionate crystal has a radioactive carbon isotope content of 20 pMC (percent modern carbon) or more, 50 pMC or more, 90 pMC or more, 100 pMC or more, and a biocarbon content of 20 wt. % or more, 50 wt. % or more, 80 wt. %, 90 wt. %, or 95 wt. %, as measured according to the standard of ASTM D6866-21.

The radioactive carbon isotope ratio (pMC) means the ratio between the radioactive carbon isotope (¹⁴C) contained in the 3-hydroxypropionate crystal acid and the radioactive carbon isotope (¹⁴C) of a modern reference standard material, and it can be greater than 100% because the nuclear testing program in the 1950s is still in effect and is not extinguished.

Further, the content of biocarbon means the content of biocarbon relative to the total carbon content contained in the 3-hydroxypropionate crystal. As this value is larger, it may correspond to an eco-friendly compound.

On the other hand, if the radioactive carbon isotope content (pMC) and biocarbon content of the 3-hydroxypropionate crystal are too low, the eco-friendly property is reduced, which may not be seen as a bio-derived material.

The crystalline state of the 3-hydroxypropionate crystal can be confirmed through peaks and the like in an X-ray diffraction (XRD) graph.

For example, the 3-hydroxypropionate crystal may exhibit peaks between crystal lattices in the 2θ value range of 8 to 22° during X-ray diffraction (XRD) analysis.

For example, when the concentrate contains magnesium hydroxide (Mg(OH)₂), and the formed 3-hydroxypropionate crystal is Mg(3HP)₂, peaks between the crystal lattices due to the bond between 3-hydroxypropionic acid and magnesium may appear in the 2θ value range of 8 to 15° during X-ray diffraction (XRD) analysis for Mg(3HP)₂. Such peaks show different results from the X-ray diffraction (XRD) analysis results for magnesium hydroxide (Mg(OH)₂) or magnesium sulfate (Mg(SO₄)). As a result of the X-ray diffraction (XRD) analysis, it can be confirmed that when a specific peak appears in the 2θ value range of 8 to 15°, Mg(3HP)₂ crystal is formed.

Specifically, during X-ray diffraction (XRD) analysis for Mg(3HP)₂, 3 or more, 4 or more, or 5 or more peaks may appear in the 2θ value range of 8 to 15°, and for example, peaks may appear in the 2θ value range of 8.2 to 9.3°, 9.5 to 11.0°, 11.2 to 12.7°, 12.9 to 13.3°, and 13.5 to 14.8°, respectively.

Further, when calcium hydroxide (Ca(OH)₂) is contained in the concentrate and the 3-hydroxypropionate crystal formed therefrom are Ca(3HP)₂, peaks between the crystal lattices due to the bond between 3-hydroxypropionic acid and calcium may appear in the 2θ value range of 10 to 22° during X-ray diffraction (XRD) analysis for Ca(3HP)₂. Such peaks show different results from the X-ray diffraction (XRD) analysis results for calcium hydroxide (Ca(OH)₂) or calcium sulfate (Ca(SO₄)). As a result of the X-ray diffraction (XRD) analysis, it can be confirmed that when a specific peak appears in the 2θ value range of 10 to 22°, Ca(3HP)₂ crystal is formed.

Specifically, during X-ray diffraction (XRD) analysis for Ca(3HP)₂, 3 or more, 5 or more, 7 or more or 9 or more peaks may appear in the 2θ value range of 10 to 22°, and for example, peaks may appear in the 2θ value range of 10.0 to 11.0°, 11.1 to 11.6°, 11.6 to 12.5°, 12.7 to 13.6°, 13.8 to 16.0°, 17.0 to 18.0°, 19.0 to 19.8°, 20.2 to 21.2°, or 21.5 to 22.0°, respectively.

On the other hand, the incident angle (θ) means the angle formed between the crystal plane and the X-ray when X-rays are irradiated on a specific crystal plane, and the peak means the point at which a first differential value (slope of the tangent line, dy/dx) is 0 on a graph where the horizontal axis (x-axis) in the x-y plane is a value of two times the incident angle (2θ) of the incident X-ray, and the vertical axis (y-axis) in the x-y plane is a diffraction intensity, wherein as the value (2θ) of two times the angle of incidence of the incident X-ray, which is the horizontal axis (x-axis), increases in the positive direction, the first differential value (slope of the tangent line, dy/dx) of two times (2θ) the incident angle of X-rays, which is the horizontal axis (x-axis), with respect to the diffraction intensity, which is the vertical axis (y-axis), changes from a positive value to a negative value.

Further, the 3-hydroxypropionate crystal may have a distance (d value) between atoms in the crystals derived from X-ray diffraction (XRD) analysis of 1.00 Å or more and 15.00 Å or less, 1.50 Å or more and 13.00 Å or less, 2.00 Å or more and 11.00 Å or less, 2.50 Å or more and 10.00 Å or less.

For example, when the 3-hydroxypropionate crystal is Mg(3HP)₂, a distance (d value) between atoms in the crystals of the peak appearing in the 2θ value range of 8 to 15° may be 1.00 Å or more and 15.00 Å or less, 2.00 Å or more and 13.00 Å or less, 4.00 Å or more and 11.00 Å or less, 5.50 Å or more and 10.00 Å or less.

Further, when the 3-hydroxypropionate crystal is Ca(3HP)₂, the distance (d value) between atoms in the crystals of the peak appearing in the 2θ value range of 10 to 22° may be 1.00 Å or more and 15.00 Å or less, 2.00 Å or more and 13.00 Å or less, 3.00 Å or more and 10.00 Å or less, 3.40 Å or more and 9.00 Å or less, or 4.00 Å or more and 8.50 Å or less.

Further, the 3-hydroxypropionate crystal may have a glass transition temperature of −55° C. or more and −30° C. or less, a melting point of 30° C. or more and 170° C. or less, and a crystallization temperature of 25° C. or more and 170° C. or less.

The glass transition temperature, melting point, and crystallization temperature may be measured by a differential scanning calorimetry (DSC) for the 3-hydroxypropionate crystal, wherein the heating rate during measurement may be 1 to 20° C./min. Further, the 3-hydroxypropionate crystal may have a glass transition temperature of −55° C. or more and −30° C. or less, −50° C. or more and −35° C. or less, or −45° C. or more and −40° C. or less. Further, the melting point of the 3-hydroxypropionate crystal may be 30° C. or more and 170° C. or less, 31° C. or more and 160° C. or less, 32° C. or more and 150° C. or less. Further, the crystallization temperature of the 3-hydroxypropionate crystal may be 25° C. or more and 170° C. or less, 27° C. or more and 160° C. or less, or 30° C. or more and 150° C. or less. In addition, the crystallization stability section of the 3-hydroxypropionate crystal may be −40° C. to 150° C.

The recovery rate of 3-hydroxypropionic acid can be calculated by a method of Equation 1 below.

$$3 HP recovery rate ( \% ) = \qquad [ Equation 1 ]$$

$$\{ ( 3 HP content in crystals ) /$$

$$( 3 HP content in fermentation liquid before crystallization ) \} * 100$$

In one example, the recovery rate of 3-hydroxypropionic acid in the process of recovering 3-hydroxypropionic acid provided by the present invention may be 40% or more, 50% or more, 60% or more, 70% or more, 80% or more, or 90% or more, for example, 40 to 99.9%, 50 to 99.9%, 60 to 99.9%, 70 to 99.9%, 80 to 99.9%, 90 to 99.9%, 40 to 99%, 50 to 99%, 60 to 99%, 70 to 99%, 80 to 99%, 90 to 99%, 40 to 97%, 50 to 97%, 60 to 97%, 70 to 97%, 80 to 97%, 90 to 97%, 40 to 95%, 50 to 95%, 60 to 95%, 70 to 95%, 80 to 95%, or 90 to 95%, but is not limited thereto. The recovery rate may be calculated on a weight basis.

Further, in the process of recovering 3-hydroxypropionic acid (3HP), the purity of 3-hydroxypropionate contained in the 3-hydroxypropionate crystal can be calculated as the percentage (%) of the weight of the compound having the Structural Formula 1 and/or 2 relative to the weight of the total crystals recovered. In one example, the purity of 3-hydroxypropionate contained in the 3-hydroxypropionate crystal produced in the recovery process of 3-hydroxypropionate provided by the present invention may be 70% or more, 80% or more, 90% or more, 70 to 99.9%, 80 to 99.9%, 90 to 99.9%, 70 to 99%, 80 to 99%, or 90 to 99%, but is not limited thereto.

Further, after the step of forming the 3-hydroxypropionate crystal, the 3-hydroxypropionate crystal can be separated from the concentrate and converted to 3-hydroxypropionic acid.

The method of separating a 3-hydroxypropionate crystal from the concentrate can be performed by selecting a method known in the art to which the present invention belongs without limitation within the scope of the purpose for separating the crystal. In one example, the recovery of the 3-hydroxypropionate crystal may be performed by drying (e.g., heat drying, etc.) and/or filtration methods, but is not limited thereto.

Further, the method of converting (purifying) the separated 3-hydroxypropionate crystal to 3-hydroxypropionic acid may be performed by selecting a method known in the art without limitation within the scope of the purpose of purifying 3-hydroxypropionic acid. For example, one or more of the methods listed below may be used, but are not limited thereto.

For example, there may be mentioned:
i) a method of removing cations through a cation exchange resin to protonate 3-hydroxypropionic acid;
ii) a method of extracting cations (e.g., Mg²⁺, Ca²⁺, etc.) with an organic solvent using a liquid cation exchange to protonate 3-hydroxypropionic acid; and
iii) a method of titrating with an acid (e.g., sulfuric acid, etc.) to produce a salt (e.g., CaSO₄(s) or MgSO₄(s)), thereby protonating 3-hydroxypropionic acid.

The 3-hydroxypropionic acid may contain a radioactive carbon isotope (¹⁴C). The radioactive carbon isotope (¹⁴C) contains about 1 atom per 10¹² carbon atoms in the earth's atmosphere, and has a half-life of about 5700 years. The stock of carbon can become abundant in the upper atmosphere by a nuclear reaction involving cosmic rays and ordinary nitrogen (¹⁴N). Meanwhile, in fossil fuels, isotopes have decayed over a long time, so that the ¹⁴C ratio may be substantially zero. When bio-derived raw material is used as a raw material for 3-hydroxypropionic acid or fossil fuel is used together with it, the content of radioactive carbon isotope (pMC; percent modern carbon) and the content of biocarbon contained in the 3-hydroxypropionic acid can be measured according to the standard of ASTM D6866-21.

As an example of the measurement method, the carbon atoms contained in the compound to be measured can be made into graphite or carbon dioxide gas, and the content can be measured using a mass spectrometer, or can be measured according to a liquid scintillation analysis method. At this time, the two radioactive isotopes can be separated using an accelerator for separating ¹⁴C ions from ¹²C ions together with the mass spectrometer, and the content and the content ratio can be measured by a mass spectrometer.

The 3-hydroxypropionic acid may have a radioactive carbon isotope content of 20 pMC (percent modern carbon) or more, 50 pMC or more, 90 pMC or more, or 100 pMC or more, and a biocarbon content of 20 wt. % or more, 50 wt. % or more, 80 wt. %, 90 wt. %, or 95 wt. % as measured according to the standard of ASTM D6866-21.

The radioactive carbon isotope ratio (pMC) means the ratio between the radioactive carbon isotope (¹⁴C) contained in the 3-hydroxypropionic acid and the radioactive carbon isotope (¹⁴C) of a modern reference standard material, and it can be greater than 100% because the nuclear testing program in the 1950s is still in effect and is not extinguished.

Further, the content of biocarbon means the content of biocarbon relative to the total carbon content contained in the 3-hydroxypropionic acid. As this value is larger, it may correspond to an eco-friendly compound.

On the other hand, if the radioactive carbon isotope content (pMC) and biocarbon content of the 3-hydroxypropionic acid are too low, eco-friendly property is reduced, which may not be seen as a bio-derived material.

The poly(3-hydroxypropionate) may be a polymer obtained by polymerizing or copolymerizing monomers containing 3-hydroxypropionic acid. The poly(3-hydroxypropionate) can exhibit eco-friendly property and biodegradability of 3-hydroxypropionic acid.

The poly(3-hydroxypropionate) has a weight average molecular weight (Mw) of 10,000 to 300,000 g/mol as measured using gel permeation chromatography (GPC). More specifically, the poly(3-hydroxypropionate) has a weight average molecular weight of 10,000 g/mol or more, 20,000 g/mol or more, or 25,000 g/mol or more, and 300,000 g/mol or less, or 200,000 g/mol or less, or 100,000 g/mol or less. If the weight average molecular weight of the poly(3-hydroxypropionate) is too small, the overall mechanical properties may be significantly reduced, and if the weight average molecular weight is too large, the process procedure may be difficult, and the processability and elongation may be lowered.

### Advantageous Effects

According to the present invention, it is possible to provide a method for producing acrylic acid in which poly(3-hydroxypropionate) is thermally decomposed in an eco-friendly and economical manner, and converted into bio-acrylic acid that can be recycled into bio-superabsorbent polymer (SAP) or bio-acrylate, in high purity and high yield.

### BRIEF DESCRIPTION OF THE DRAWINGS

FIG. 1 is a graph showing the results of thermogravimetric analysis of poly(3-hydroxypropionate) under a zinc oxide catalyst.

FIG. 2 is a graph showing the results of thermogravimetric analysis of poly(3-hydroxypropionate) under a titanium oxide catalyst.

### DETAILED DESCRIPTION OF THE EMBODIMENTS

Hereinafter, the present invention will be described in more detail with reference to examples. However, the following examples are for illustrative purposes only, and are not intended to limit the scope of the present invention.

### Production Example 1: Production of poly(3-hydroxypropionate)

A BtuR gene encoding adenosyltransferase was cloned into plasmid pCDF containing a gene (dhaB) encoding glycerol dehydratase, a gene (aldH) encoding aldehyde dehydrogenase and a gene (gdrAB) encoding glycerol dehydratase reactivase. The resulting pCDF_J23101_dhaB_gdrAB_J23100_aldH_btuR vector was introduced into strain W3110 (KCCM 40219) by an electroporation method using an electroporation device (Bio-Rad, Gene Pulser Xcell) to prepare 3-hydroxypropionic acid-producing strain. The process of preparing the 3-hydroxypropionic acid-producing strain of Preparation Example 1 and the vectors, primers, and enzymes used were carried out with reference to Example 1 of Korean Unexamined Patent Publication No. 10-2020-0051375, which is incorporated herein by reference.

The 3-hydroxypropionic acid-producing strain was fermented and cultured at 35° C. in a 5 L fermenter using unpurified glycerol as a carbon source to produce 3-hydroxypropionic acid. In order to prevent the lowering of pH due to the production of 3-hydroxypropionic acid, calcium hydroxide (Ca(OH)₂), which is an alkali metal salt, was added thereto to maintain the pH to be neutral during the fermentation. After fermentation culture, cells were removed by centrifugation (4000 rpm, 10 minutes, 4° C.), and primary fermentation liquid purification (primary purification) was performed using activated carbon. Specifically, activated carbon was added to the fermentation liquid from which bacterial cells were removed by centrifugation, the mixture was well mixed, and then centrifuged again to separate the activated carbon. Then, the fermentation liquid from which the activated carbon was separated was filtered with a vacuum pump through a 0.7 um filter paper to purify the 3-hydroxypropionic acid fermentation liquid.

The concentration of 3-hydroxypropionic acid in the fermentation liquid after completion of the primary purification was a level of 50 to 100 g/L, and the fermentation liquid was concentrated to a concentration of 600 g/L using a rotary evaporator (50° C., 50 mbar) to prepare a concentrate, and stirred (300 rpm) at room temperature to produce Ca(3HP)₂ crystals. At this time, the concentration of the alkali metal salt in the concentrate was 493.3 g/L (based on Ca(OH)₂). The resulting crystals were washed three times with ethanol (EtOH) and dried in an oven at 50° C. to finally recover the crystals. Cations were removed through a cation exchange resin, and 3-hydroxypropionic acid was protonated, recovered and purified.

25 ml of an 60% 3-hydroxypropionic acid aqueous solution was added to a 100 ml Schlenk flask in an oil bath, and moisture in 3-hydroxypropionate was removed at 50° C. and 50 mbar for 3 hours, and then oligomerized at 70° C. and 20 mbar for 2 hours. Then, 0.4 parts by weight of p-toluenesulfonic acid (p-TSA) catalyst based on 100 parts by weight of 3-hydroxypropionate was added to a reaction flask, and a melt condensation polymerization reaction was performed at a temperature of 110° C. for 24 hours. After the reaction was completed, the reaction product was dissolved in chloroform, and the extracted with methanol to obtain poly(3-hydroxypropionate) (weight average molecular weight: 26,000 g/mol).

### Production Example 2: Production of poly(3-hydroxypropionate)

Poly (3-hydroxypropionate) (weight average molecular weight: 28,000 g/mol) was obtained in the same manner as Production Example 1, except that the melt polycondensation reaction was performed for 30 hours, instead of the melt polycondensation reaction for 24 hours.

### Production Example 3: Production of poly(3-hydroxypropionate)

Poly (3-hydroxypropionate) (weight average molecular weight: 21,000 g/mol) was obtained in the same manner as Production Example 1, except that the melt polycondensation reaction was performed for 16 hours, instead of the melt polycondensation reaction for 24 hours.

### Example 1

5 g of poly(3-hydroxypropionate) produced in Production Example 1, 150 mg of zinc oxide (ZnO), and 10 mg of hydroquinone monoethyl ether (MEHQ) were added to a flask, and mixed with stirring. Then, the mixture was heated to 180° C. to dissolve poly(3-hydroxypropionate). After confirming complete dissolution, the reaction temperature was raised to 240° C., and then acrylic acid coming from distillation was recovered. After completion of the reaction, a total of 4.2 g of acrylic acid was recovered (recovery rate: 84.0%).

### Example 2

Acrylic acid was recovered in the same manner as in Example 1, except that 50 mg of zinc oxide (ZnO) was used instead of 150 mg of zinc oxide (ZnO). After completion of the reaction, a total of 3.8 g of acrylic acid was recovered (recovery rate: 76%).

### Example 3

Acrylic acid was recovered in the same manner as in Example 1, except that 250 mg of zinc oxide (ZnO) was used instead of 150 mg of zinc oxide (ZnO). After completion of the reaction, a total of 4.1 g of acrylic acid was recovered (recovery rate: 82%).

### Example 4

Acrylic acid was recovered in the same manner as in Example 1, except that 500 mg of zinc oxide (ZnO) was used instead of 150 mg of zinc oxide (ZnO). After completion of the reaction, a total of 4.0 g of acrylic acid was recovered (recovery rate: 80%).

### Example 5

Acrylic acid was recovered in the same manner as in Example 1, except that 5 g of poly(3-hydroxypropionate) produced in Production Example 2 was used instead of 5 g of poly(3-hydroxypropionate) produced in Production Example 1. After completion of the reaction, a total of 4.3 g of acrylic acid was recovered (recovery rate: 86%).

### Example 6

Acrylic acid was recovered in the same manner as in Example 1, except that 5 g of poly(3-hydroxypropionate) produced in Production Example 3 was used instead of 5 g of poly(3-hydroxypropionate) produced in Production Example 1. After completion of the reaction, a total of 4.1 g of acrylic acid was recovered (recovery rate: 82%).

### Comparative Example 1

2.0 g of poly(3-hydroxypropionate) produced in Production Example 1 and 8.6 mg of hydroquinone mono ethyl ether (MEHQ) were added, and heated to 210° C. Then, 1.39 g of acrylic acid coming from distillation was recovered.

### Comparative Example 2

5.0 g of poly(3-hydroxypropionate) produced in Production Example 1, 50 mg of pentamethyl diethylene triamine, and 10 mg of hydroquinone mono ethyl ether (MEHQ) was added to a flask and mixed with stirring. The reaction temperature was raised to 80° C., and the mixture was stirred for 2 hours. At this time, melting of poly(3-hydroxypropionate) was not observed. Then, the temperature inside the reaction was raised to 290° C., and then acrylic acid coming from distillation was recovered. After completion of the reaction, a total of 3.8 g of acrylic acid was recovered (recovery rate: 76%).

### Comparative Example 3

Acrylic acid was recovered in the same manner as in Example 1, except that zinc oxide (ZnO) was not used, and thermal decomposition was performed at 290° C. instead of 240° C. After completion of the reaction, a total of 3.25 g of acrylic acid was recovered (recovery rate: 65%).

### Comparative Example 4

Acrylic acid was recovered in the same manner as in Example 1, except that 50 mg of titanium oxide (TiO₂) was used instead of 150 mg of zinc oxide (ZnO), and thermal decomposition was performed at 290° C. instead of 240° C. After completion of the reaction, a total of 3.1 g of acrylic acid was recovered (recovery rate: 62%).

### Comparative Example 5

Acrylic acid was recovered in the same manner as in Example 1, except that 150 mg of titanium oxide (TiO₂) was used instead of 150 mg of zinc oxide (ZnO), and thermal decomposition was performed at 290° C. instead of 240° C. After completion of the reaction, a total of 3.3 g of acrylic acid was recovered (recovery rate: 66%).

### Comparative Example 6

Acrylic acid was recovered in the same manner as in Example 1, except that 250 mg of titanium oxide (TiO₂) was used instead of 150 mg of zinc oxide (ZnO), and thermal decomposition was performed at 290° C. instead of 240° C. After completion of the reaction, a total of 3.0 g of acrylic acid was recovered (recovery rate: 60%).

### Comparative Example 7

Acrylic acid was recovered in the same manner as in Example 1, except that 500 mg of titanium oxide (TiO₂) was used instead of 150 mg of zinc oxide (ZnO), and thermal decomposition was performed at 290° C. instead of 240° C. After completion of the reaction, a total of 3.2 g of acrylic acid was recovered (recovery rate: 64%).

### Evaluation

### 1. Evaluation of Recovery Rate of Bio-Acrylic Acid or Acrylic Acid

The recovery rate (mol yield) of acrylic acid produced in Examples and Comparative Examples was calculated, and the results are shown in Table 1 below.

### 2. Evaluation of Purity of Bio-Acrylic Acid or Acrylic Acid

The purity of acrylic acid produced in Examples and Comparative Examples was calculated using ¹H NMR (400 MHz, CDCl₃), and the results are shown in Table 1 below.

### 3. Evaluation of Biocarbon Content of Acrylic Acid

The biocarbon content of acrylic acid produced in Examples and Comparative Examples was analyzed using ASTM D 6866-21 (Method B), and the results are shown in Table 1 below. On the other hand, if acrylic acid contains almost no biocarbon, it is indicated as ‘-’.

### 4. Thermogravimetric Analysis

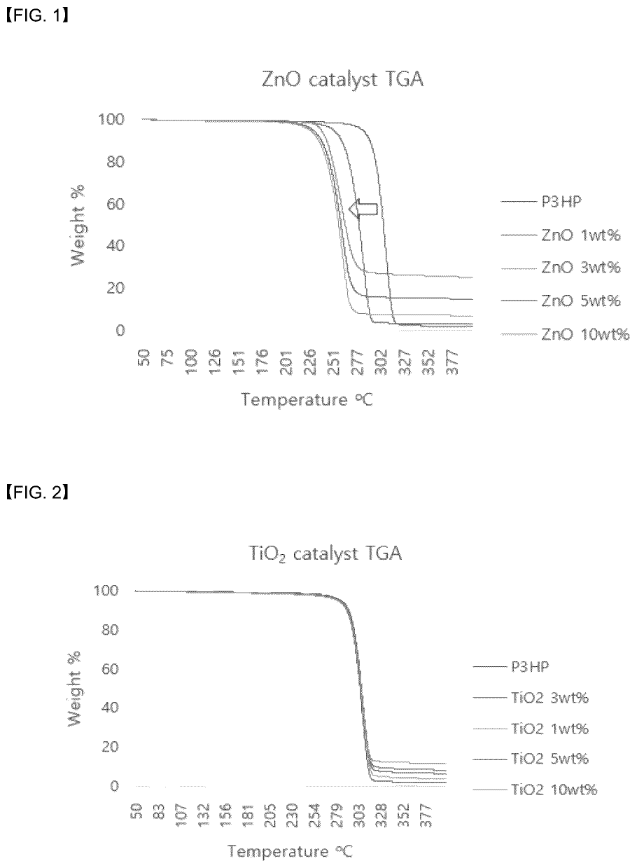

In Examples and Comparative Examples, the weight loss temperature of poly(3-hydroxypropionate) was analyzed by thermogravimetry, and the results are shown in Table 1 below. At this time, the weight loss temperature means the temperature at which the weight of poly(3-hydroxypropionate) begins to decrease. On the other hand, the thermogravimetric analysis was performed by raising the temperature from 50° C. to 400° C. at a rate of 10° C./min under a nitrogen gas (N₂) atmosphere. On the other hand, FIG. 1 is a graph showing the results of thermogravimetric analysis of poly(3-hydroxypropionate) under a zinc oxide catalyst, and FIG. 2 is a graph showing the results of thermogravimetric analysis of poly(3-hydroxypropionate) under a titanium oxide catalyst.

### 5. Measurement of Complex Viscosity

The complex viscosity of the melted (dissolved) poly(3-hydroxypropionate) (P3HP) of Examples 5 and 6 was measured, and shown in Table 2 below. The measurement was performed using a strain control rheometer ARES from TA Instruments, and the measurement was performed while changing the angular frequency from 0.1 to 500.0 rad/s at a temperature of 90° C.

TABLE 1

| | Recovery rate | Purity of | Biocarbon | Weight loss |
|---|---|---|---|---|
| | of acrylic | acrylic | content | temperature |
| | acid (%) | acid (%) | (wt. %) | (Onset, ° C.) |
| Example 1 | 84.0 | 99.0 | 100 | 246 |
| Example 2 | 76.0 | 99.0 | 100 | 267 |
| Example 3 | 82.0 | 99.0 | 100 | 245 |
| Example 4 | 80.0 | 99.0 | 100 | 244 |
| Example 5 | 86.0 | 99.0 | 100 | — |
| Example 6 | 82.0 | 99.0 | 100 | — |
| Comparative | 69.5 | 99.4 | 0 | — |
| Example 1 | | | | |
| Comparative | 76.0 | 87.0 | 100 | — |
| Example 2 | | | | |
| Comparative | 65.0 | 99.0 | 100 | 295 |
| Example 3 | | | | |
| Comparative | 62.0 | 98.0 | 100 | 297 |
| Example 4 | | | | |
| Comparative | 66.0 | 94.0 | 100 | 297 |
| Example 5 | | | | |
| Comparative | 60.0 | 98.0 | 100 | 297 |
| Example 6 | | | | |
| Comparative | 64.0 | 95.0 | 100 | 297 |
| Example 7 | | | | |

According to Table 1, it was confirmed that Example 1 is significantly superior to Comparative Example 1 in the recovery rate of acrylic acid. In particular, it was confirmed that Comparative Example 1 has a low recovery rate of 69.5% as thermal decomposition proceeds without a melting process. In addition, it was confirmed that Comparative Example 1 has a biocarbon content of 0 due to the use of petrochemical-based poly(propiolactone). On the other hand, it was confirmed that in Comparative Example 2, the polymer is heated at 80° C. for 2 hours, but the polymer is not melted, and that when the polymer is thermally decomposed at 290° C., the pentamethyl diethylene triamine catalyst is also vaporized and mixed with the product acrylic acid, thereby significantly reducing the purity of acrylic acid.

Further, it was confirmed that Comparative Examples 4 to 7 using titanium oxide as a catalyst have a high weight loss temperature, and thermal decomposition was not performed well at 240° C., and so the recovery rate of acrylic acid was low.

It was confirmed that Examples 1 to 4, in which zinc oxide was used as a catalyst, have a low weight loss temperature, allow good thermal decomposition at 240° C., and recover acrylic acid at a high recovery rate.

TABLE 2

| Angular | Complex viscosity | Complex viscosity |
|---|---|---|
| frequency | (Pa · s) of melted | (Pa · s) of melted |
| (rad/s) | P3HP of Example 5 | P3HP of Example 6 |
| 500 | — | 11.6718 |
| 281.171 | 22.9903 | 11.8526 |
| 158.114 | 23.2557 | 11.9152 |
| 88.914 | 23.2979 | 11.9003 |
| 50 | 23.2601 | 11.8735 |
| 28.1171 | 23.1668 | 11.8037 |
| 15.8114 | 23.0823 | 11.7197 |
| 8.8914 | 23.0088 | 11.6802 |
| 5 | 22.8861 | 11.6294 |
| 2.81171 | 22.9252 | 11.6327 |
| 1.58114 | 22.8694 | 11.5508 |
| 0.88914 | — | 11.5336 |
| 0.5 | — | 11.8044 |

According to Table 2, it was confirmed that the melted poly(3-hydroxypropionate) has a complex viscosity of 11.5336 to 23.2979 Pa·s at angular frequencies of 0.5 to 500 rad/s.

1. A method for producing acrylic acid comprising thermally decomposing a poly(3-hydroxypropionate) in the presence of a transition metal oxide of one of Groups 5 to 12 of the periodic table of elements to produce the acrylic acid.

2. The method for producing acrylic acid according to claim 1,
further comprising melting the poly(3-hydroxypropionate) before the thermal decomposition step.

3. The method for producing acrylic acid according to claim 2, wherein:
the melting is performed at a temperature of 150° C. or more and 200° C. or less.

4. The method for producing acrylic acid according to claim 1, wherein:
the thermal decomposition is performed at a temperature of 200° C. or more and 250° C. or less.

5. The method for producing acrylic acid according to claim 2, wherein:
a difference between the melting temperature and the thermal decomposition temperature is 20° C. or more and 130° C. or less.

6. The method for producing acrylic acid according to claim 2, wherein:
the poly(3-hydroxypropionate) melted by the melting has a complex viscosity of 5.0 Pa·s or more and 30.0 Pa·s or less at an angular frequency of 0.1 to 500.0 rad/s.

7. The method for producing acrylic acid according to claim 2, wherein:
the melting is performed under a solvent-free condition.

8. The method for producing acrylic acid according to claim 1, wherein:
the thermal decomposition is performed under a solvent-free condition.

9. The method for producing acrylic acid according to claim 1, wherein:
the transition metal oxide of one of Groups 5 to 12 of the periodic table of elements is one or more selected from the group consisting of zinc oxide, iron oxide, copper oxide, nickel oxide, cobalt oxide, manganese oxide, chromium oxide, and molybdenum oxide.

10. The method for producing acrylic acid according to claim 1, wherein:
the transition metal oxide of one of Groups 5 to 12 of the periodic table of elements is used in an amount of 0.01 parts by weight or more and 30 parts by weight or less based on 100 parts by weight of the poly(3-hydroxypropionate).

11. The method for producing acrylic acid according to claim 1, wherein:
the acrylic acid has a biocarbon content of 80 wt. % or more as measured by the standard of ASTM 6866-21.

12. The method for producing acrylic acid according to claim 1,
further comprising recovering the acrylic acid by distillation under reduced pressure.

13. The method for producing acrylic acid according to claim 2, wherein
before the melting the poly(3-hydroxypropionate),
the method further comprises polymerizing 3-hydroxypropionic acid to produce the poly(3-hydroxypropionate).

14. The method for producing acrylic acid according to claim 13, wherein
the 3-hydroxypropionic acid is produced by fermenting a strain having 3-hydroxypropionic acid production ability.

15. The method for producing acrylic acid according to claim 1, wherein
the method for producing acrylic acid recycles biodegradable articles comprising the poly(3-hydroxypropionate).
